(19) United States
(12) Patent Application Publication
(10) Pub. No.: US 2003/0040680 A1
(43) Pub. Date: Feb. 27, 2003

(54) Eye blinking bio-feedback apparatus and method

(75) Inventors: Richard A. Hassert, Wheaton, IL (US); Suzanne L. Hassert, Wheaton, IL (US)

Correspondence Address: SONNENSCHEIN NATH & ROSENTHAL, P.O. BOX 061080, WACKER DRIVE STATION, CHICAGO, IL, 60606-1080, US

(73) Assignee: Clear View Scientific, LLC

(21) Appl. No.: 09/938,138

(22) Filed: Aug. 23, 2001

(51) Int. Cl. A61B 5/04; A61B 13/00

(52) U.S. Cl. 600/558; 600/545

(57) ABSTRACT

An eye blinking bio-feedback device and method which produces an eye blink in a user of a computer display through a sensory stimulus occurring at a quasi-random rate.

### BACKGROUND OF THE INVENTION

The present invention relates to bio-feedback methods and devices.

Computer vision syndrome (CVS) is a repetitive stress injury to the eyes that can result from use of a computer display. The symptoms of CVS are dry, red, painful eyes, often accompanied by difficulty focusing, and headaches. Additional symptoms include heaviness of the user's eyelids, changes in color perception, neck and shoulder pain, backaches, and muscle spasms. If left untreated, CVS can lead to musculoskeletal disorders in the neck and shoulders. For example, computer users run the risk of straining their necks or awkwardly positioning their backs and shoulders in order to clearly see a computer display. Studies estimate that around 70-91% of the people who use computers for more than two hours a day suffer from CVS.

While the symptoms are most often associated with the use of cathode ray tube (CRT) displays, many users report symptoms of CVS from the use of liquid crystal displays (LCD) on laptop computers.

The causes of CVS are not fully understood, however it is believed that CVS results from overuse of a computer display, constant refocusing, glare, poor lighting, and the phenomenon of reduced blinking while using a computer display.

It is believed that the phenomenon of reduced blinking while using a computer display is the primary cause of CVS. Blinking is an eye's way of re-lubricating and relaxing itself. A blink distributes moisture from the crease of the eye over the entire surface of the eyelid. A person normally blinks about twenty two times per minute. According to studies, computer users blink only seven times per minute. This is because computer users tend to stare at computer displays, which reduces their frequency of blinking. The problem is aggravated by the use of contact lenses and certain medicines such as antihistamines.

Several approaches have been taken to address CVS. Products such as task lighting, background lighting, anti-glare filters, computer glasses, and eye drops have been introduced to address the problem of CVS. Further, it has been recommended to have a clean computer display and to maintain an appropriate distance between a user and a computer display.

While these products and methods are important to addressing CVS, if a person uses a computer display for two or more hours in a day and does not blink at a rate of about 12 blinks per minute, the person will probably still suffer CVS. Once the eye is traumatized, rest and eye drops are needed to provide relief. And if a computer display is used daily, it is difficult for the user to get enough rest for their eyes. On the other hand, if the user does blink at a rate of about 12 blinks per minute, even without strong lighting and without a glare filter, eye trauma is minimized.

### SUMMARY OF THE INVENTION

The present disclosure provides one or more inventions directed to reminding a user of a computer display to blink. These inventions can be practiced jointly or separately.

To this end, in an embodiment, there is provided an eye blinking bio-feedback device which produces an eye blink in a user of a computer display through a sensory stimulus occurring at a quasi-random rate. The eye blinking bio-feedback device can be mounted, for example, on the computer display.

There is also provided, in an embodiment, an eye blinking bio-feedback device comprising a signal generator for generating a quasi-random signal; and at least one indicator for producing a sensory indication based on the quasi-random signal.

The sensory indication can be, for example, at least one of light, sound, and a puff of air. Further, when the sensory indication is light, the indicator can comprise at least two illuminating indicators. These two illuminating indicators can be spaced apart by, for example, three inches.

In an embodiment, the sensory indication is produced at a rate of 12 or more times per minute, and preferably at a rate of from 12 to 26 times per minute based on the quasi-random signal.

There is also provided, in an embodiment, a method for producing an eye blink in a user of a computer display. An eye blinking bio-feedback device comprising: a signal generator for generating a quasi-random signal; and at least one indicator for producing a sensory indication based on the quasi-random signal is provided to the user. The user is trained to blink when the user senses the sensory indication produced by the indicator.

In an embodiment, the training step further comprises: producing the sensory indication based on the quasi-random signal; the user blinking when the user senses the sensory indication; and repeating the producing and blinking steps until the user unconsciously blinks when the user senses the sensory indication.

There is also provided, in an embodiment, a method for producing an eye blink in a user of a computer display. The method comprises the step of providing an eye blinking bio-feedback device comprising a signal generator for generating a quasi-random signal, and at least one indicator for producing a sensory indication based on the quasi-random signal, the user blinking when the user senses the sensory indication produced by the indicator.

There is also provided, in an embodiment, a bio-feedback method for producing an eye blink in a user of a computer display. An eye blinking bio-feedback device is provided that comprises a signal generator for generating a quasi-random signal, and at least one indicator for producing a sensory indication based on the quasi-random signal. The quasi-random signal is generated. The user blinks when the user senses the sensory indication produced by the indicator.

In accordance with methods consistent with the present invention, there is provided a method in a data processing system for producing an eye blink in a user of a computer display. The method comprises the steps of providing an eye blinking bio-feedback device comprising a signal generator for generating a quasi-random signal and at least one indicator for producing a sensory indication based on the quasi-random signal, and training the user to blink when the user senses the sensory indication produced by the indicator.

In accordance with systems consistent with the present invention, there is provided a data processing system comprising: a memory comprising a computer program that provides an eye blinking bio-feedback device comprising a signal generator for generating a quasi-random signal, and at least one indicator for producing a sensory indication based on the quasi-random signal, and trains the user to blink when the user senses the sensory indication produced by the indicator; and a processing unit that runs the computer program.

In accordance with articles of manufacture consistent with the present invention, there is provided a computer-readable medium containing instructions that cause a data processing system to perform a method for producing an eye blink in a user of a computer display. The method comprises the steps of: providing an eye blinking bio-feedback device comprising: a signal generator for generating a quasi-random signal, and at least one indicator for producing a sensory indication based on the quasi-random signal; and training the user to blink when the user senses the sensory indication produced by the indicator.

These and other features of the present invention will become clearer with reference to the following detailed description of the presently preferred embodiments and accompanying drawings.

### DESCRIPTION OF THE DRAWINGS

FIG. 1 depicts a schematic perspective view of an eye blinking bio-feedback device in accordance with an embodiment of the present invention.

FIG. 2 depicts a schematic perspective view of a person using a computer monitor and the eye blinking bio-feedback device depicted in FIG. 1.

FIG. 3 depicts a block diagram of the eye-blinking bio-feedback device depicted in FIG. 1.

FIG. 4 depicts a graphical representation of eye blink frequency of a person using the eye blinking bio-feedback device depicted in FIG. 1 in accordance with methods of the present invention.

FIG. 5 depicts a block diagram of a data processing system with which embodiments of the present invention may be implemented.

FIG. 6 depicts a block diagram of a client-server based data processing system with which embodiments of the present invention may be implemented.

DETAILED DESCRIPTION OF THE PRESENTLY PREFERRED EMBODIMENTS

As discussed above, there are provided methods and devices to remind a person to blink.

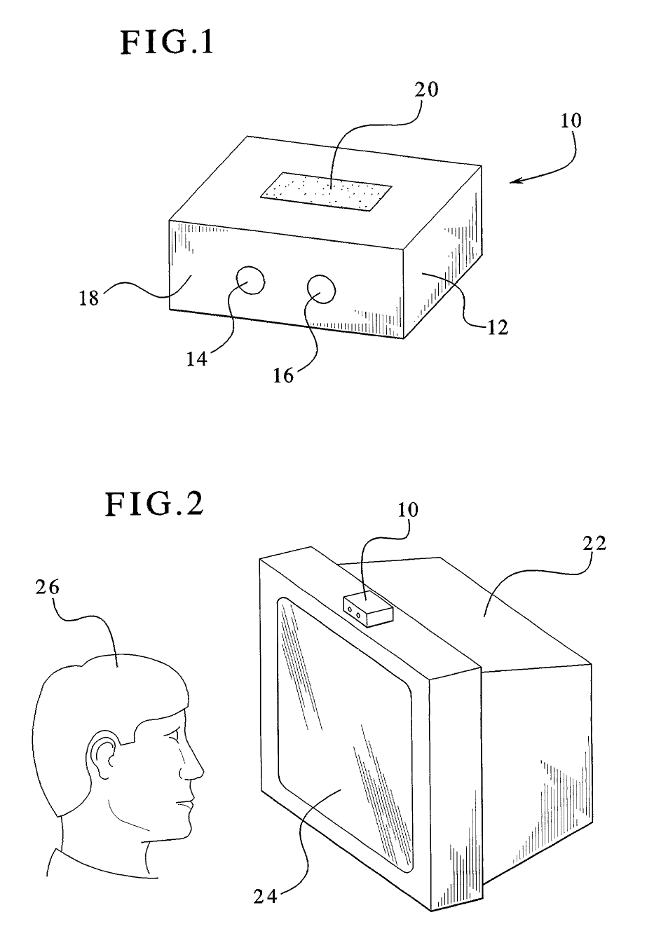

FIG. 1 depicts a schematic perspective view of an eye blinking bio-feedback device 10 in accordance with an embodiment of the present invention. The eye blinking bio-feedback device 10 has a first indicator 14 and a second indicator 16 located on a front face 18 of an enclosure 12.

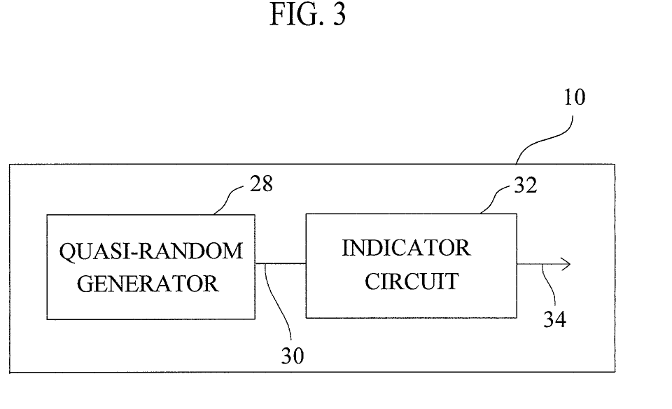

Referring to FIG. 3, the eye blinking bio-feedback device 10 comprises a quasi-random signal generator 28 connected via at least one electrical coupling 30 to an indicator circuit 32. The indicator circuit 32 is connected via at least one electrical connection 34 to the first indicator 14 and the second indicator 16.

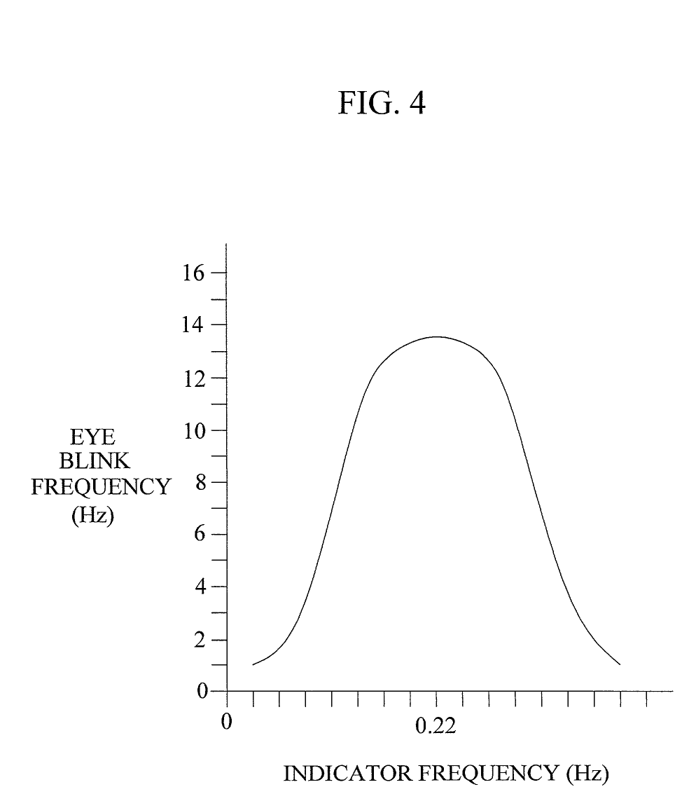

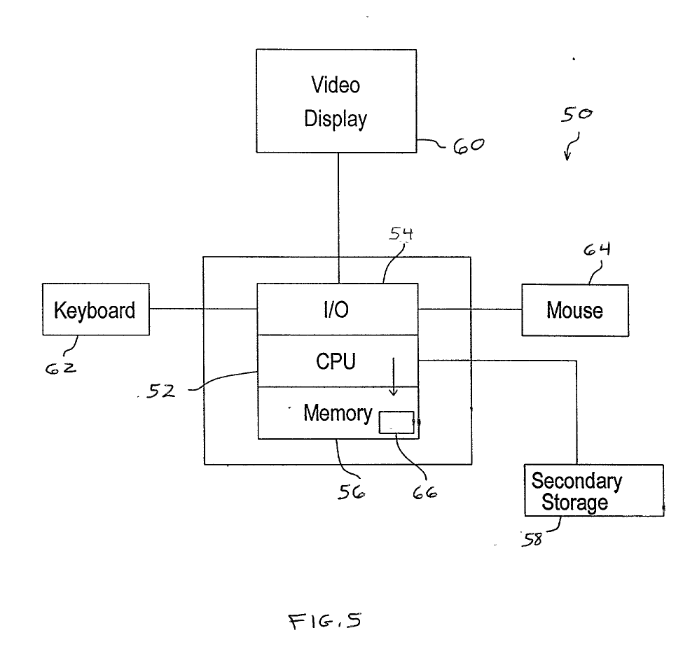

The quasi-random signal generator 28 generates at least one quasi-random signal that has a duration and frequency which mimics a typical human blinking pattern. A human typically blinks at a rate of about 12 blinks per minute. Referring to FIG. 4, the quasi-random signal generator 28 generates a quasi-random signal having a bell-shaped distribution with a median frequency of 0.22 Hz or about 12 per minute.

The quasi-random generator 28 can comprise any means for generating a controlled quasi-random signal. For example, the quasi-random generator 28 can comprise a pulse generator, which is controlled to randomly output pulses at a rate of from about 12 to about 26 times per minute. Control of the pulse generator can be performed by the pulse generator itself or via a controller.

The quasi-random signal is transmitted via the electrical coupling 30 to the indicator circuit 32, where at least one output signal is output via at least one output signal line 34 to the first indicator 14 and the second indicator 16 based on the quasi-random signal. The output signal can comprise a respective output signal for each of the indicators 14 and 16. This provides independent quasi-random signals to the first indicator 14 and the second indicator 16.

Alternatively, the first indicator 14 and the second indicator 16 can be connected directly to the quasi-random signal generator 28 via the electrical coupling 30. Accordingly, the electrical coupling can comprise a respective electrical coupling 30 for each indicator 14 and 16.

As depicted in FIG. 2, the eye blinking bio-feedback device 10 can be provided on a computer display 22 wherein the device 10 is located such that the user 26 will receive a sensory stimulus from the device 10 while the user 26 is viewing a screen 24 of the computer display 22. The eye blinking bio-feedback device 10 can be mounted on the computer display 22 with a mounting device 20. The mounting device 20 can be, for example, an adhesive device or a magnetic device.

It is believed that phenomenon of reduced blinking while using a computer display is the primary cause of CVS. A person normally blinks about twenty two times per minute. According to studies, users of computer displays blink only seven times per minute. This is because users of computer displays tend to stare at the computer displays, which reduces their frequency of blinking. The users essentially forget to blink.

Methods and devices in accordance with the present invention inventively help to prevent CVS by training the user to blink while using a computer display. The eye blinking bio-feedback device 10 is provided on the computer display 22 that the user 26 is using. While user 26 is staring at the screen 24 of the computer display 22, the quasi-random signal generator 28 generates quasi-random signals which respectively drive the first indicator 14 and second indicator 16 at quasi-random rates of greater than about 12 times per minute, and preferably from about 12 to about 26 times per minute. Each time the first indicator 14 or second indicator 16 receives an appropriate signal level of the quasi-random signal, the first indicator 14 or second indicator 16 will produce a sensory indication.

The sensory indication can be of any type that will produce a sensory stimulus in the user 26. The sensory indication can be, for example, optical, acoustic, or motional events, or any combination of these events. When the sensory indication is an optical event, it can be, for example, an illumination of light from the indicator 14 or 16. When the sensory indication is an acoustic event, it can be, for example, a sound from the indicator 14 or 16. And when the sensory indication is a motional event, it can be, for example, a puff of air.

The sensory indication occurs with a frequency based on the quasi-random signal and at a rate that mimics the blinking pattern of a normal person. Thus, the sensory indication will occur at a quasi-random rate of preferably from about 12 to about 26 times per minute. The sensory indication will maintain in the “ON” state for a time period of from about 0.1 second to about 1 second.

The example eye blinking bio-feedback device 10 depicted in FIGS. 1 and 2 has a first indicator 14 and a second indicator 16. Alternatively, the eye blinking bio-feedback device 10 can have any number of indicators. These indicators can be driven to produce a sensory indication either concurrently or independently of each other. For example, the first indicator 14 and the second indicator 16 can each be driven by a different quasi-random signal, and therefore blink independently of each other.

The user 26 initially trains its mind to blink each time a sensory indication is produced by one of the indicators 14 and 16. As the sensory indications are produced at a rate that mimics the blinking frequency of a normal person, the user 26 who blinks in accordance with the sensory indication will blink at a normal blinking frequency. Through repeated training, the user's 26 mind will be trained to blink each time the sensory indication is produced. Once the user's 26 mind has been trained in this manner, the user 26 will no longer have to purposefully think about blinking each time a sensory indication is produced. Instead, the user 26 will be trained to blink each time a sensory indication is produced. Accordingly, once the user 26 has been trained, the user 26 will blink at a normal quasi-random rate of greater than about 12 times per minute, and preferably at a rate of about 12 to about 26 times per minute while using the computer display 22.

The amount of time that is required to train a user will vary for each user. Further, the user 26 may require retraining if the has not used the eye blinking bio-feedback device 10 for an extended period of time.

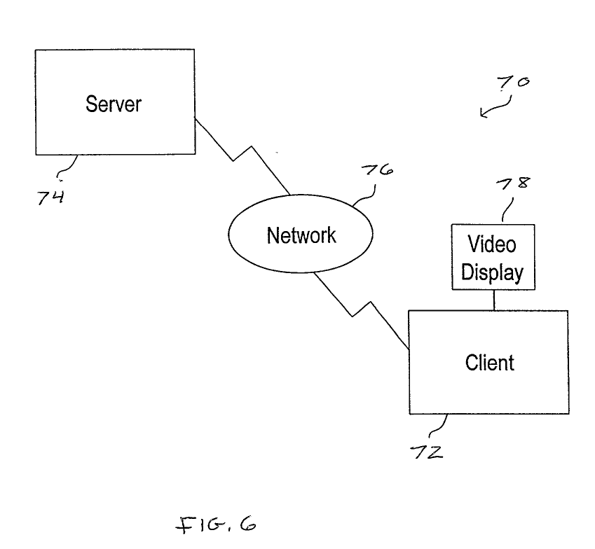

One skilled in the art will appreciate that methods consistent with the present invention may also be implemented as a computer program. The computer program can be implemented in, for example, a data processing system, line the one depicted in FIG. 5.

FIG. 5 depicts a block diagram of a data processing system 50 suitable for practicing methods and implementing systems consistent with the present invention. The data processing system 50 comprises a central processing unit (CPU) 52, an input output I/O unit 54, a memory 56, a secondary storage device 58, and a video display 60. The data processing system 50 may further comprise standard input devices such as a keyboard 62, a mouse 64 or a speech processing means (not illustrated).

The memory 56 contains a computer program 66. The computer program 66 can comprise a plurality of computer programs that are implemented in hardware or software. Computer program 66 provides an interface for presenting a sensory indication based on a quasi-random signal for producing an eye blink in the user of the data processing system 50 and for making user selections via the keyboard 62. The sensory indication based on the quasi-random signal is, for example, viewed on the video display 60 or heard from a speaker (not shown) of the data processing system 50. For example, the sensory indication can be an alteration of the illuminated display of the video display 60, such as illuminating a plurality of pixels on the video display 60.

Although aspects of one implementation are depicted as being stored in memory, one skilled in the art will appreciate that all or part of systems and methods consistent with the present invention may be stored on or read from other computer-readable media, such as secondary storage devices, like hard disks, floppy disks, and CD-ROM; a carrier wave received from a network such as the Internet; or other forms of ROM or RAM. Further, although specific components of data processing system 50 are been described, one skilled in the art will appreciate that a data processing system suitable for use with methods, systems, and articles of manufacture consistent with the present invention may contain additional or different components.

One skilled in the art will also appreciate that methods, systems, and articles of manufacture consistent with the present invention may also be implemented in a client-server environment, like the one depicted in FIG. 6. FIG. 6 depicts a block diagram of a client-server based data processing system 70 with which methods, systems, and articles of manufacture consistent with the present invention may be implemented. A client computer system 72 and a server computer system 74 are each connected to a network 76, such as a Local Area Network, Wide Area Network, or the Internet. An sensory indication based on a quasi-random signal is displayed, for example, on a video display 78 of the client computer system 72 while some or all steps of the processing as described below are carried out on the server computer system 74, which is accessed by the client computer system 72 over the network 76.

In accordance with methods, systems, and articles of manufacture consistent with the present invention, an eye blink can be produced in a user of a video display based on a sensory indication produced by the data processing system 50. Computer program 66 generates a quasi random signal which enables the sensory indication. The quasi-random signal generated by the computer program 66 is similar to the quasi-random signal generated by the quasi-random signal generator 28, which was described above.

The sensory indication can be, for example, an illumination of light from the video display 60 or an acoustic event emitted from a speaker (not shown) of the data processing system. The sensory indication is similar to the sensory indication described above with reference to FIGS. 1-4, and similarly the user is trained to blink when the user senses the sensory indication.

The foregoing provides methods and devices for significantly reducing eye fatigue and pain in a user of a computer display. Further, as the user is trained to blink at a normal blinking frequency, the sensory indications are not overly distracting, are not monotonous, should not cause drowsiness, and should not set off a seizure. The methods and devices in accordance with the present invention advantageously prevent CVS.

As is apparent from the foregoing specification, the present invention is susceptible to being embodied with various alterations and modifications which may differ particularly from those that have been described in the preceding specification and description. It should be understood that it is desired to embody within the scope of the patent warranted herein all such modifications as reasonably and properly come within the scope of the presently defined contribution to the art.

We claim as our invention:

1. An eye blinking bio-feedback device which produces an eye blink in a user of a computer display through a sensory stimulus occurring at a quasi-random rate.

2. An eye blinking bio-feedback device comprising:
a signal generator for generating a quasi-random signal; and
at least one indicator for producing a sensory indication based on the quasi-random signal.

3. The device of claim 2, wherein the sensory indication is an optical event.

4. The device of claim 2, wherein the sensory indication is an acoustic event.

5. The device of claim 2, wherein the sensory indication is a motion event.

6. The device of claim 2, wherein the indicator comprises at least two illuminating indicators.

7. The device of claim 6, wherein the at least two illuminating indicators are spaced about three inches apart.

8. The device of claim 2, wherein the sensory indication is produced at a rate of greater than about 12 times per minute based on the quasi-random signal.

9. The device of claim 2, wherein the sensory indication is produced at a rate of from about 12 to about 26 times per minute based on the quasi-random signal.

10. The device of claim 2, wherein the device is mounted on the computer display.

11. A method for producing an eye blink in a user of a computer display, the method comprising the steps of:
providing an eye blinking bio-feedback device comprising:
a signal generator for generating a quasi-random signal; and
at least one indicator for producing a sensory indication based on the quasi-random signal; and
training the user to blink when the user senses the sensory indication produced by the indicator.

12. The method of claim 11, wherein the training step further comprises:
producing the sensory indication based on the quasi-random signal;
the user blinking when the user senses the sensory indication; and
repeating the producing and blinking steps until the user unconsciously blinks when the user senses the sensory indication.

13. The method of claim 11, wherein the sensory indication is an optical event.

14. The method of claim 11, wherein the sensory indication is an acoustic event.

15. The method of claim 11, wherein the sensory indication is a motion event.

16. The method of claim 11, wherein the indicator comprises at least two illuminating indicators.

17. The method of claim 16, wherein the at least two illuminating indicators are spaced about three inches apart.

18. The method of claim 11, wherein the sensory indication is produced at a rate of greater than about 12 times per minute based on the quasi-random signal.

19. The method of claim 11, wherein the sensory indication is produced at a rate of from about 12 to about 26 times per minute based on the quasi-random signal.

20. A method for producing an eye blink in a user of a computer display, the method comprising the steps of:
providing an eye blinking bio-feedback device comprising:
a signal generator for generating a quasi-random signal; and
at least one indicator for producing a sensory indication based on the quasi-random signal, the user blinking when the user senses the sensory indication produced by the indicator.

21. A bio-feedback method for producing an eye blink in a user of a computer display, the method comprising the steps of:
providing an eye blinking bio-feedback device comprising:
a signal generator for generating a quasi-random signal; and
at least one indicator for producing a sensory indication based on the quasi-random signal;
generating the quasi-random signal; and
the user blinking when the user senses the sensory indication produced by the indicator.

22. The method of claim 21, wherein the sensory indication is an optical event.

23. The method of claim 21, wherein the sensory indication is an acoustic event.

24. The method of claim 21, wherein the sensory indication is a motion event.

25. The method of claim 21, wherein the indicator comprises at least two illuminating indicators.

26. The method of claim 25, wherein the at least two illuminating indicators are spaced about three inches apart.

27. The method of claim 21, wherein the sensory indication is produced at a rate of greater than about 12 times per minute based on the quasi-random signal.

28. The method of claim 21, wherein the sensory indication is produced at a rate of from about 12 to about 26 times per minute based on the quasi-random signal.

29. A method in a data processing system for producing an eye blink in a user of a computer display, the method comprising the steps of:
providing an eye blinking bio-feedback device comprising:
a signal generator for generating a quasi-random signal; and
at least one indicator for producing a sensory indication based on the quasi-random signal; and
training the user to blink when the user senses the sensory indication produced by the indicator.

30. A data processing system comprising:
a memory comprising a computer program that provides an eye blinking bio-feedback device comprising a signal generator for generating a quasi-random signal, and at least one indicator for producing a sensory indication based on the quasi-random signal, and trains the user to blink when the user senses the sensory indication produced by the indicator; and
a processing unit that runs the computer program.

31. A computer-readable medium containing instructions that cause a data processing system to perform a method for producing an eye blink in a user of a computer display, the method comprising the steps of:
providing an eye blinking bio-feedback device comprising:
a signal generator for generating a quasi-random signal; and
at least one indicator for producing a sensory indication based on the quasi-random signal; and
training the user to blink when the user senses the sensory indication produced by the indicator.
